(19) United States
(12) Patent Application Publication
(10) Pub. No.: US 2015/0335869 A1
(43) Pub. Date: Nov. 26, 2015

(54) OCULAR TREATMENT USING MEDICATED ABSORBENT PAD

(71) Applicant: William Agnew, Salisbury, MD (US)

(72) Inventor: William Agnew, Salisbury, MD (US)

(21) Appl. No.: 14/282,774

(22) Filed: May 20, 2014

(51) Int. Cl. A61M 35/00

(52) U.S. Cl. CPC A61M 35/00

(57) ABSTRACT

A sterile cotton fiber pad to deliver generic prescription medication transdermally through the eyelid is provided. The sterile absorbent pad may contain liquid medication and may be applied to the thin, highly vascularized tissue of the eyelids by direct application.

### BACKGROUND OF THE INVENTION

The present invention relates to ocular treatment and, more particularly, to ocular treatment using a medicated absorbent pad.

Ocular drug delivery includes the delivery of medications to the eye to treat an assortment of eye diseases. A common method of ocular drug delivery is eye drops. Eye drops may include saline-containing drops used as an ocular route to administer. Depending on the condition being treated, they may contain steroids, antihistamines, sympathomimetics, beta receptor blockers. parasympathomimetics, parasympatholytics, prostaglandins, non-steroidal anti-inflammatory drugs, antibiotics, antifungal, or topical anesthetics. Eye drops are typically delivered using eye droppers. For some individuals, dropping the solution into the eye may be difficult. For example, the user may have a disability, such as tremors, that would prevent the user from accurately dropping the solution into the eye. Further, the user may rapidly blink from discomfort and therefore may not focus on holding the bottle in the proper position.

As can be seen, there is a need for an improved method of treating the eye.

### SUMMARY OF THE INVENTION

In one aspect of the present invention, a method of medication delivery comprises: providing an absorbent cotton fiber pad; applying a liquid medication to the absorbent cotton fiber pad, thereby allowing the liquid medication to absorb into the absorbent cotton fiber pad; applying the absorbent cotton fiber pad with the absorbed liquid medication to a patient's eyelid.

In another aspect of the present invention, a treatment device comprises: an absorbent cotton fiber pad; and a liquid ophthalmic medication absorbed within the absorbent cotton fiber pad.

These and other features, aspects and advantages of the present invention will become better understood with reference to the following drawings, description and claims.

### BRIEF DESCRIPTION OF THE DRAWINGS

FIG. 1 is a perspective view of the absorbent pad of the present invention within packaging;

FIG. 2 is a perspective view of the absorbent pad removed from the packaging;

FIG. 3 is a cross sectional view along line 3-3 in FIG. 2 illustrating the medication within the absorbent pad; and

FIG. 4 is the absorbent pad of the present invention in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes the use of a sterile cotton fiber pad to deliver generic prescription medication transdermally through the eye lid. The sterile absorbent pad may contain medication and may be applied to the thin, highly vascularized tissue of the eyelids by direct application. The present invention may deliver a generic Rx ophthalmic medication to the user's eye without the use of an eye dropper. Therefore, a user with a disability may easily apply the pad to the eyelid and deliver the medication.

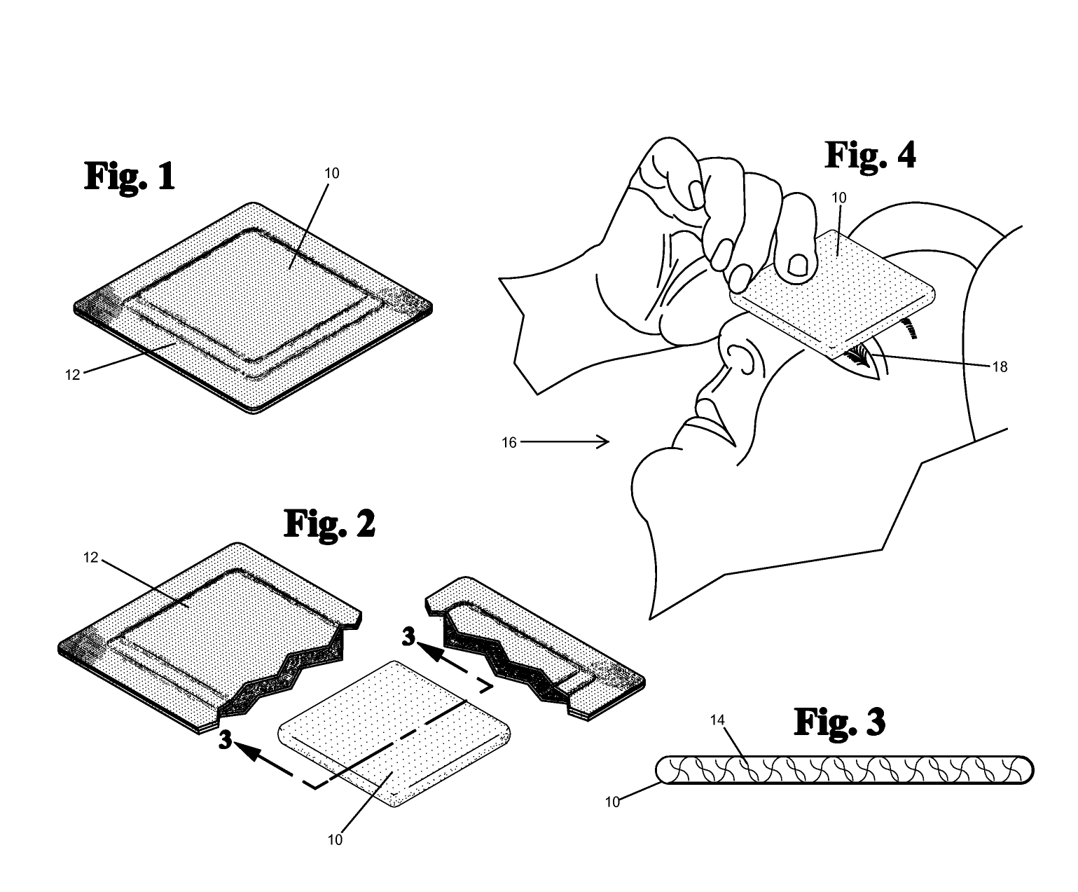

Referring to FIGS. 1 through 4, the present invention includes an absorbent cotton fiber pad 10. The absorbent cotton fiber pad 10 may be stored within a packaging 12 so that the absorbent cotton fiber pad 10 remains sterile while not in use. The absorbent cotton fiber pad 10 may be removed from the packaging 12 prior to use. Once the absorbent cotton fiber pad 10 has been removed, a liquid medication 14 may be applied to the pad 10. The absorbent cotton fiber pad 10 may absorb the medication 14 and retain the medication 14 within the cotton fibers.

The liquid medication 14 of the present invention may be a liquid ophthalmic medication, including, but not limited to, steroids, antihistamines, sympathomimetics, beta receptor blockers, parasympathomimetics, parasympatholytics, prostaglandins, non-steroidal anti-inflammatory drugs, antibiotics, antifungal, or topical anesthetics. Once the medication 14 has been applied to the absorbent cotton fiber pad 10, a user 16 may apply the pad 10 to the user's eyelid 18. In certain embodiments, the user 16 may close their eyelid 18 and apply the absorbent cotton fiber pad 10 with the medication 14 onto the eyelid 18 so that the medication may transdermally pass through the eyelid 18 and treat the eye of the user 16. Placing a pad 10 onto the eyelid 18 may be a lot easier for the user 16 as compared to applying eye drops to an eye via an eyedropper.

A method of medication delivery may include the following. An absorbent cotton fiber pad 10 may be provided. The liquid medication 14 may be applied to the absorbent cotton fiber pad 10, thereby allowing the liquid medication 14 to absorb into the absorbent cotton fiber pad 10. The absorbent cotton fiber pad 10 with the absorbed liquid medication may be applied to a patient's eyelid 18. In certain embodiments, a sterile absorbent cotton fiber pad 10 may be removed from a packaging 12 prior to applying the liquid medication 14 to the absorbent cotton fiber pad 10. The liquid medication 14 may be a liquid ophthalmic medication 14. The absorbent cotton fiber pad 10 with the liquid medication 14 may be applied to the patient's eyelid 18 while the patient's eyelid is closed over the patient's eye.

It should be understood, of course, that the foregoing relates to exemplary embodiments of the invention and that modifications may be made without departing from the spirit and scope of the invention as set forth in the following claims.

What is claimed is:

1. A treatment device comprising:
an absorbent cotton fiber pad; and
a liquid ophthalmic medication absorbed within the absorbent cotton fiber pad.

2. A method of medication delivery comprising:
providing an absorbent cotton fiber pad;
applying a liquid medication to the absorbent cotton fiber pad, thereby allowing the liquid medication to absorb into the absorbent cotton fiber pad;
applying the absorbent cotton fiber pad with the absorbed liquid medication to a patient's eyelid.

3. The method of claim 1, wherein the absorbent cotton fiber pad is sterile.

4. The method of claim 2, further comprising the step of removing the sterile absorbent cotton fiber pad from a packaging prior to applying the liquid medication to the absorbent cotton fiber pad.

5. The method of claim 1, wherein the liquid medication is a liquid ophthalmic medication.

6. The method of claim 1, further comprising the step of holding the absorbent cotton fiber pad with the liquid medication to the patient's eyelid while the patient's eyelid is closed.
